(19) United States
(12) Patent Application Publication
(10) Pub. No.: US 2006/0194344 A1
(43) Pub. Date: Aug. 31, 2006

(54) Plasmon resonant structure, controlling method thereof, and a metallic domain manufacturing method

(76) Inventor: Takao Saito, Gunma (JP)

Correspondence Address: KNOBBE MARTENS OLSON & BEAR LLP, 2040 MAIN STREET, FOURTEENTH FLOOR, IRVINE, CA, 92614, US

(21) Appl. No.: 11/339,964

(22) Filed: Jan. 25, 2006

(30) Foreign Application Priority Data
Jan. 25, 2005 (JP) 2005-017510

(51) Int. Cl. C12M 1/34; G01N 1/28; G01N 33/543

(52) U.S. Cl. 436/518; 427/2.11; 435/287.2

(57) ABSTRACT

Metallic particle layers with metallic domains being arranged therein each at a predetermined space within a horizontal plane are laminated at an appropriate distance in the vertical direction in a dielectric layer. The distance ΔW between each of the metallic domains may be controlled by controlling the growth of metallic particles for the horizontal direction and the distance ΔL between the metallic particle layers may be controlled by controlling the thickness of the dielectric layer to be laminated for the vertical direction, so that the effect of field enhancement by plasmon resonance is improved by satisfactory control for the plasmon resonance in the direction of the thickness and in the direction orthogonal thereto.

### BACKGROUND OF THE INVENTION

1. Field of the Invention

The present invention concerns a plasmon resonance structure, a controlling method thereof and a metallic domain manufacturing method and, more specifically, it relates to plasmon resonance control.

2. Description of the Related Art

In near field optics, it has been devised to unitize the effect of field enhancement, etc. by utilizing surface plasmon resonance and studies have been made on applications of various fields such as communication and recording media (Applied Physics, Vol 73, No. 10 (2004) “Propagation of Spreading of Surface Plasmon Polariton and Control”, p 1275-1284). For the effect of field enhancement, fine particles of from several nm to several hundreds of nm (hereinafter referred to as “nanoparticles”) are formed and the localized surface plasmon generated in the vicinity thereof is utilized. The nanoparticles are usually formed by a chemical method, for example, a sol-gel method and used in a state dispersed three dimensionally in a film. FIG. 7 shows an example in which nanoparticles 902 of a metal are dispersed at random in a dielectric film.

### SUMMARY OF THE INVENTION

In the localized surface Plasmon resonance, when light is applied to nanoparticles in the direction of the film thickness, it is distributed in the orthogonal direction. Accordingly, for controlling the localized surface plasmon resonance, separate modes should be considered within the plane in the direction of the thickness and in the direction orthogonal to the thickness. However, in a plasmon resonant film obtained by the chemical method described above, nanoparticles are in a state dispersed at random in a three-dimensional manner and the plasmon resonance can not be controlled by separate modes in the direction of the film thickness and the direction orthogonal thereto. Accordingly, the effect of field enhancement by the plasmon has not been used efficiently.

In view of the foregoing, the present invention intends to favorably control the plasmon resonance in the direction of the thickness of a structure and a direction orthogonal thereto. The invention further intends to control the plasmon resonance and improve the effect of filed enhancement thereof.

For attaining the object described above, the present invention includes forming a plurality of metallic particle layers containing nanoparticles or metallic domains in a dielectric material, and controlling the plasmon resonance by:

(1) controlling a distance between each of the metallic particle layers,

(2) controlling a space between metallic particles contained in each of the metallic particles layer, and/or

(3) both of (1) and (2) above. The foregoing and other objects, features and advantages of the invention will become apparent from the following detailed description and accompanying drawings.

According to the invention, the plasmon resonance is controlled by controlling the distance between the metal particle layers in the direction of the thickness of the dielectric film, and the space between the metal particles in the direction perpendicular to the direction of the thickness, respectively.

Accordingly, plasmon resonance can be controlled favorably and the effect of field enhancement can be improved.

### BRIEF DESCRIPTION OF THE DRAWINGS

FIGS. 1A to 1D are cross sectional views showing a lamination structure and main manufacturing process of a plasmon resonance structure in Example 1 of the invention;

FIG. 2 is a graph showing the absorbance in each of samples of Experimental Example 1 in the example described above;

FIGS. 3A, 3B are graphs showing the absorbance in each of samples of Experimental Example 2 in the example described above;

FIG. 4 is a graph showing the absorbance in each of samples of Experimental Example 3 in the example described above;

FIG. 5 is a graph showing the absorbance in each of samples of Experimental Example 4 in the example described above;

FIG. 6 is a graph showing the absorbance in each of samples of Experimental Example 5 in the example described above;

FIG. 7 is a cross sectional view showing the laminate structure of the prior art and that of Example 2 of the invention.

### DESCRIPTION OF PREFERRED EMBODIMENTS

Preferred embodiments of the present invention are to be described specifically with reference to the examples.

### EXAMPLE 1

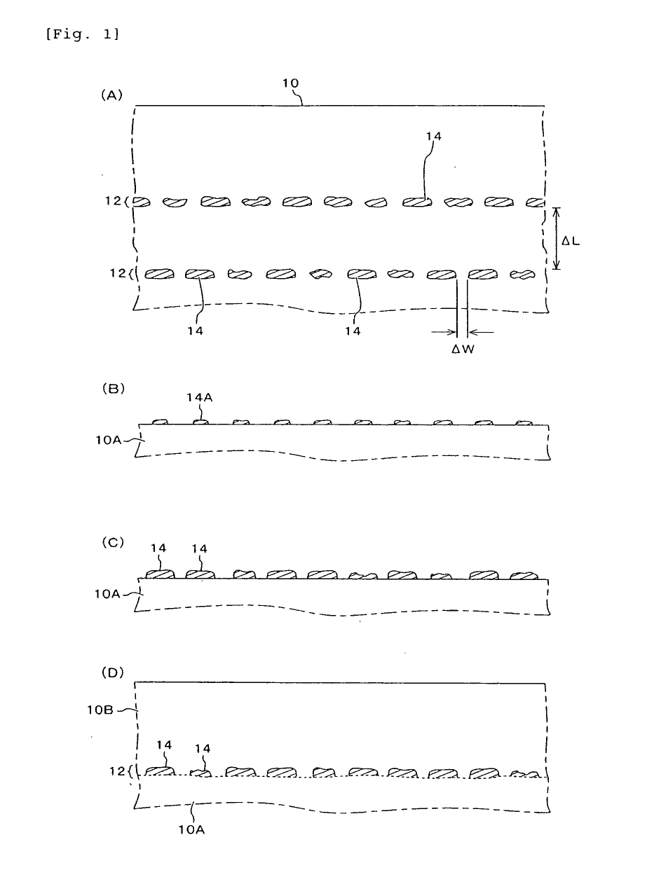

Example 1 of the invention is to be described at first with reference to FIG. 1 to FIG. 6. FIG. 1A shows a cross sectional structure of a plasmon resonance structure in this example. As shown in the drawing, a dielectric layer 10 has a structure in which layers 12 of nanoparticles or metallic domains 14 (hereinafter both of them are collectively referred to as “metallic domain”) are laminated each at an appropriate distance in the horizontal direction of the drawing. The metallic particle layer 12 has a constitution in which the metallic domains 14 are arranged being spaced apart from each other within a horizontal plane. As the dielectric layer 10, SiO₂ is used for example. Further, as the metallic domain 14, a metal such as Au, Ag, or Al can be used.

While known methods may be used for forming the domain structure, it is formed, for example, by the method shown in FIGS. 1B to 1D. At first, as shown in FIG. 1B, metallic particles 14A for an SiO₂ layer 10A are formed on the main surface of the SiO₂ layer 10A, for example, by sputtering. In the initial stage for forming the film, the metallic film is not formed over the entire main surface but the metallic particles 14A are deposited in an island state. As the sputtering proceeds further, the metallic particles 14A grow on the main surface as shown in FIG. 1(C) to form metallic domains 14. Then, as shown in FIG. 1(D), an SiO₂ layer 10B is formed over the metallic domains 14. By conducting the treatment described above repetitively, a plasmon resonant structure shown in FIG. 1(A) can be obtained. Since the SiO₂ layers 10A and 10B are made of an identical material, they form a structure as if the metallic domains 14 “floated” in the SiO₂ layer 10.

As described above, the example has a structure in which

(1) the metallic domains 14 are arranged being spaced from each other within a horizontal plane to form the metallic particle layer 12, and

(2) metallic particle layers 12 are positioned with respect to each other at a predetermined distance in the vertical direction in the dielectric layer 10. Accordingly, the space ΔW between the metallic domains 14 can be controlled by controlling the growth of the metallic particle 14A for the horizontal direction. Further, the distance ΔL between the metallic particle layers 12 can be controlled by controlling the thickness of the dielectric layer 10B laminated in the vertical direction.

Then, after manufacturing the sample as described above, the plasmon resonance was measured. The plasmon resonance was measured by irradiating a spread light to the sample and measuring the absorbance in the sample by a spectrophotometer. This is because the absorbance changes when the spread light is converted to the plasmon depending on the degree of the conversion.

### (1) Experimental Example 1

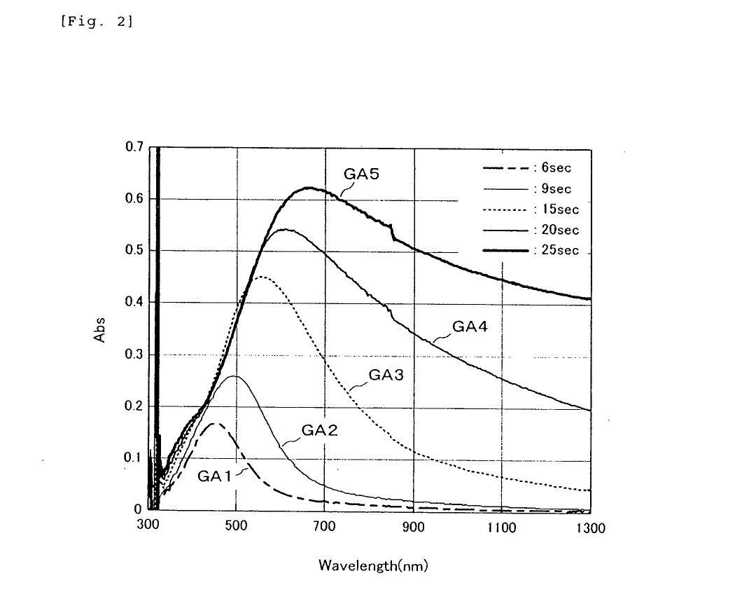

A single-layer of Ag particles was used as the metallic particles layer 12, which was formed by one layer in the dielectric layer of SiO₂. Then, when the absorbance was measured by the method described above, a result as shown in FIG. 2 was obtained. In the figure, the abscissa indicates a wavelength (indicated as “Wavelength”) and the ordinate indicates the absorbance (indicated as “Abs”). Further, in graphs GA1 to GA5, the sputtering time was changed stepwise from 6 sec to 25 sec. The sputtering time corresponded to the domain size and as the sputtering time was longer, the domain grew more and the space ΔW between the metallic domains 14 decreased.

As shown in FIG. 2, a peak appeared for the absorbance in each of the cases and it is considered that the peak is the loss of the spread light due to the generation of the plasmon and the plasmon resonance is controlled by the structure in this example. Further, the peak for the absorbance is shifted to a longer wavelength zone as the size of the metallic domain 14 was larger and the resonance wavelength band of the plasmon can be controlled by controlling the size of the metallic domain 14. Further, since the inter-particle space changed in this case, the peak intensity could also be controlled.

### (2) Experimental Example 2

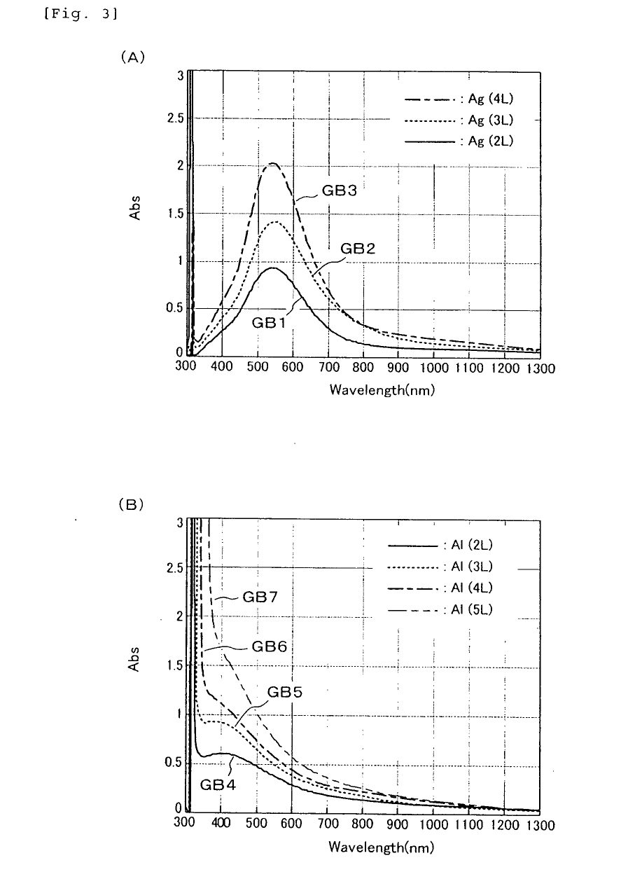

An Ag or Al particle layer was used as the metallic particle layer 12, which was formed in plurality with a distance: ΔL=80 nm therebetween in the dielectric layer of SiO₂. Then, when the absorbance was measured in the same manner, a result as shown in FIG. 3 was obtained. Graphs GB1 to GB3 shown in FIG. 3(A) are the cases using Ag for the metallic domain 14 in which metallic particle layer 12 are formed in two layers, three layers, and four layers, respectively. In the same manner, graphs GB4 to GB7 shown in FIG. 3(B) are the cases using Al for the metallic domain 14 in which metallic particle layer 12 are formed in two layers, three layers, and four layers and five layers, respectively.

At first, taking notice on the case of Ag in FIG. 3(A), as a result of the design intended for the absorbance of from 540 to 550 nm, the absorbance of the metallic particle layer 12 was 0.93 for the two layers, 1.42 for the three layers, and 2.03 for the four layers. When comparing the graphs GB1 to GB3 to each other, the absorbance increased as the number of lamination layers was larger and it can be seen that the plasmon intensity is improved by the increase in the number of layers of the metallic domain 14.

In the case of Al in FIG. 3(B), as shown in the graphs GB4 to GB7, the peak for the absorbance near 400 nm increased as the number of the lamination layers increased. Thus, it can be seen that a sufficient plasmon resonance can be obtained also by the use of inexpensive Al instead of expensive Ag. Particularly, in the graph GB7 for the number of lamination layers of 5, an absorbance as high as 1.63 was obtained in a 405 nm zone corresponding to a so-called blue laser.

### (3) Experimental Example 3

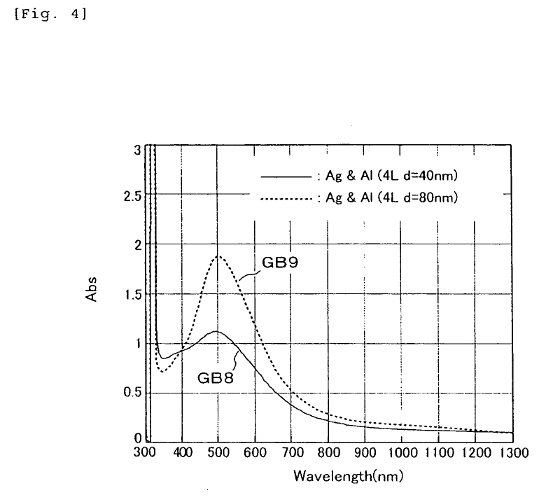

Then, description is to be made with reference to FIG. 4 to a case of forming Ag metallic particle layers 12 and Al metallic particle layers 12 alternately by two layers each, that is, by four layers in total while changing the distance ΔL. The distance ΔL is defined as below:

a: graph GB8: ΔL=40 nm,

b: graph GB9: ΔL=80 nm.

When both of them are compared, it can be seen that the peak for the absorbance is higher as the distance ΔL is larger and the effect of forming the plasmon is higher. However, the position for the peak is different between Ag and Al and it can be seen that the wavelength characteristics can be controlled finely by combination of different materials. On the other hand, since the peak intensity can be controlled by the number of the metallic particle layers 12 as described above, the wavelength characteristics and the peak intensity can be designed freely by further applying the control for the size of the metallic domains 14 or applying the control due the combination of the materials thereto.

### (4) Experimental Example 4

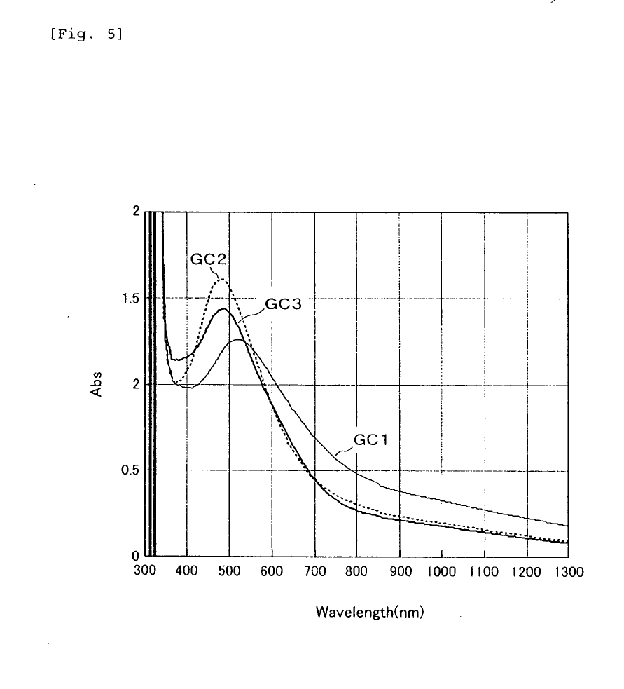

While an identical material was used for the laminated metallic particle layers 12 in each of the examples described above, identical effect can be obtained also by using different materials on every layer. FIG. 5 shows a case of forming the metallic particle layers 12 by the number of five and using the following materials for each of the layers:

a: graph GC1: Ag/Ag/Al/Al/Al,

b: graph GC2: Al/Ag/Al/Ag/Al,

c: graph GC3: Ag/Al/Al/Al/Ag, respectively.

Since the peak positions change also in the graphs GCI to GC3 respectively, it can be seen that the wavelength characteristics and the amount of resonance can be controlled also by the combination of the materials.

### (5) Experimental Example 5

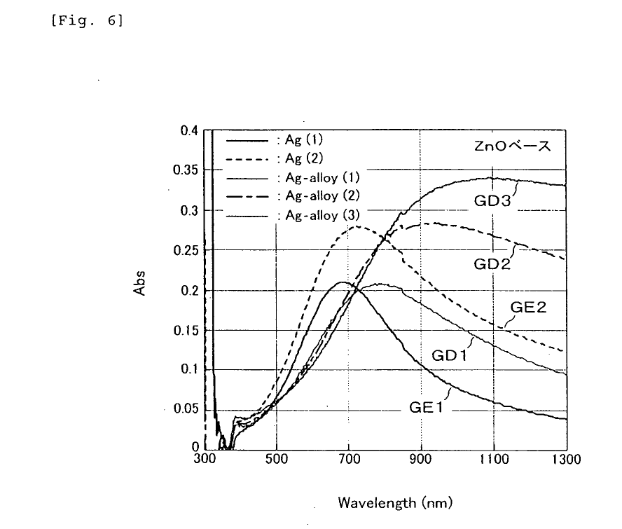

While elemental materials were used for the laminated metallic particle layers 12 in each of the examples above, the same effect can be obtained also by using alloys. FIG. 6 shows an example of using an Ag alloy as the material in the domain growing process in a case where the metallic particle layer 12 was formed in one layer. In the graphs GD1 to GD3, the domain space and the domain size are different. Also from the comparison of the graphs, the same effect as that in the previous examples can be obtained. Graphs GE1 and GE2 show the case of using only Ag.

Summarizing the results of the experiments described above:

a: As the space ΔW for the metallic domains 14 is narrower, the absorbance is higher and, as the size is larger, the peak is shifted to longer wavelengths.

b: Within a range of ΔL of 100 nm or less, as the distance ΔL of the metallic particle layers 12 is larger, the absorbance is higher.

c: As the number of layers of the metallic particle layer 12 is larger, the absorbance is higher.

By the utilization of the features described above the plasmon resonance in the direction of the thickness and in the direction orthogonal thereto can be controlled favorably thereby improving the effect of field enhancement by the plasmon resonance.

### EXAMPLE 2

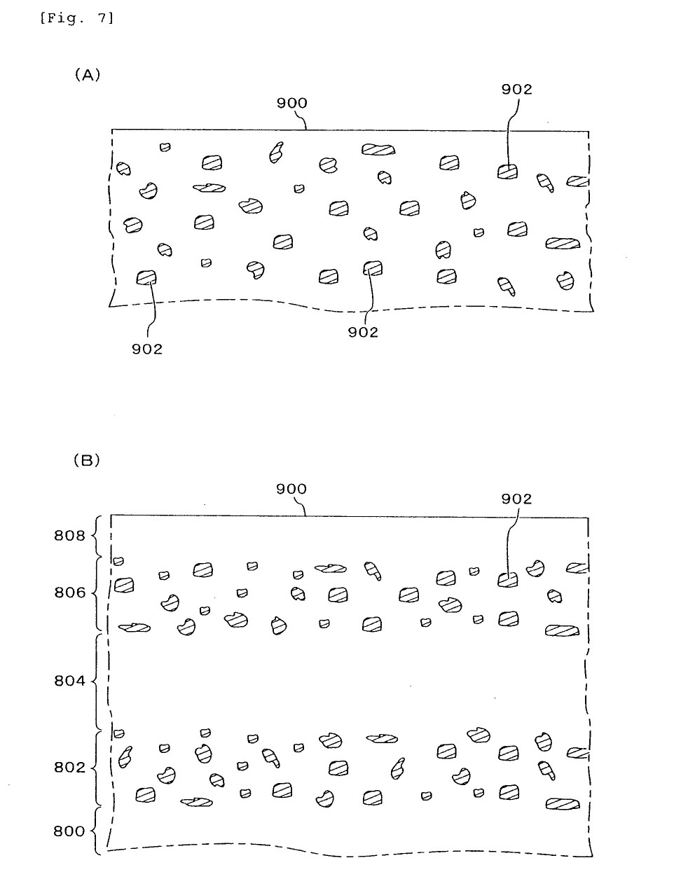

Then, Example 2 of the invention is to be described with reference to FIG. 7. In this example, an existent plasmon resonant structure using a sol-gel method shown in FIG. 7(A) was formed by lamination at a predetermined distance in a dielectric film as shown in FIG. 7(B). That is, a plasmon resonant layer 802 by a sol-gel method was formed over a dielectric layer 800 and, further, a dielectric layer 804, a plasmon resonance layer 806, and a dielectric layer 808 were formed successively thereover by lamination to prepare a plasmon resonant structure. An effect due to the multi-layered structure can be utilized by changing the distance between the plasmon resonant layers 802 and 806.

The present invention is not restricted to the examples described above but can be modified variously within a range not departing from the gist of the invention.

According to the invention, since the plasmon resonance in the direction of the thickness of the plasmon resonance structure and in the direction orthogonal thereto can be controlled favorably to improve the effect of field enhancement, the invention is suitable to various kinds of sensors and optical circuit elements, etc.

What is claimed is:

1. A method of controlling plasmon resonance including;
forming a plurality of metallic particle layers in a dielectric material, and
controlling the plasmon resonance by controlling a distance between at least two of the metallic particle layers, thereby controlling the plasmon resonance.

2. A method of controlling plasmon resonance including;
forming one or more metallic particle layers in a dielectric material, and
controlling the plasmon resonance by controlling the space between at least some metallic particles contained in at least one metallic particle layer.

3. A method of controlling plasmon resonance including;
forming a plurality of metallic particle layers in a dielectric material, and
controlling the plasmon resonance by controlling both a distance between at least some of the metallic particle layers and a space between at least some of the metallic particles contained in at least one metallic particle layer.

4. A method of controlling the plasmon resonance according to claim 1, wherein at least some metallic particles in a first layer comprise a different material than at least some metallic particles in a second layer.

5. A method of controlling the plasmon resonance according to claim 2, wherein at least some metallic particles in a first layer comprise a different material than at least some metallic particles in a second layer.

6. A method of controlling the plasmon resonance according to claim 3, wherein at least some metallic particles in a first layer comprise a different material than at least some metallic particles in a second layer.

7. A plasmon resonant structure in which a plurality of metallic particle layers are formed in a dielectric material, wherein a distance between at least two of the metallic particle layers is set to a predetermined value for obtaining a desired plasmon resonance.

8. A plasmon resonance structure in which at least one metallic particle layer is formed in a dielectric material, wherein a space between at least some of the metallic particles contained in at least one metallic particle layer is set to a predetermined value for obtaining a desired plasmon resonance.

9. A plasmon resonance structure in which a plurality of metallic particle layers are formed in a dielectric material, wherein a distance between at least two of the metallic particle layers is set to a predetermined value for obtaining a desired plasmon resonance, and wherein a space between at least some metallic particles contained in at least one metallic particle layer is set to a predetermined value for obtaining a desired plasmon resonance.

10. A plasmon resonance structure according to claim 7, wherein a dielectric material in which metallic particles are dispersed at random is used as at lest one metallic particle layer.

11. A plasmon resonance structure according to claim 9, wherein a dielectric material in which metallic particles are dispersed at random is used as at lest one metallic particle layer.

12. A method of controlling the plasmon resonance according to claim 7, wherein at least some metallic particles in a first layer comprise a different material than at least some metallic particles in a second layer.

13. A method of controlling the plasmon resonance according to claim 8, wherein at least some metallic particles in a first layer comprise a different material than at least some metallic particles in a second layer.

14. A method of controlling the plasmon resonance according to claim 9, wherein at least some metallic particles in a first layer comprise a different material than at least some metallic particles in a second layer.

15. A method of controlling the plasmon resonance according to claim 10, wherein at least some metallic particles in a first layer comprise a different material than at least some metallic particles in a second layer.

16. A method of manufacturing metallic domains by controlling an island state of the metallic domains by sputtering.

17. A plasmon resonance structure containing metallic domains manufactured by the method of manufacturing metallic domains according to claim 10.

18. A method of manufacturing a plasmon resonant structure comprising:
(a) depositing a set of metallic particles at desired locations and separated by desired inter-particle spacings on a substrate;
(b) depositing a dielectric layer of a desired thickness over said metallic particles; and
(c) repeating steps (a) and (b) at least one additional time each.
